(19) United States
(12) Patent Application Publication
(10) Pub. No.: US 2013/0041022 A1
(43) Pub. Date: Feb. 14, 2013

(54) Glyceollins Suppress Androgen-Responsive Prostate Cancer

(71) Applicants: Thomas E. Cleveland, Mandeville, LA (US); Stephen M. Boue, New Orleans, LA (US); Matthew E. Burow, Slidell, LA (US); Thomas T.Y. Wang, Potomac, MD (US)

(72) Inventors: Thomas E. Cleveland, Mandeville, LA (US); Stephen M. Boue, New Orleans, LA (US); Matthew E. Burow, Slidell, LA (US); Thomas T.Y. Wang, Potomac, MD (US)

(73) Assignees: UNITED STATES DEPARTMENT OF AGRICULTURE, Beltsville, MD (US); THE ADMINISTRATORS OF THE TULANE EDUCATIONAL FUND, New Orleans, LA (US)

(21) Appl. No.: 13/141,572

(22) PCT Filed: Dec. 22, 2009
(86) PCT No.: PCT/US09/69250

§ 371 Date: May 15, 2012

Related U.S. Application Data
(60) Provisional application No. 61/203,415, filed on Dec. 23, 2008.

(51) Int. Cl. A61K 31/35; C07D 493/14; A61P 35/00; C12N 5/09

(52) U.S. Cl. 514/453; 435/375; 549/382

(57) ABSTRACT

The present disclosure demonstrates the molecular effects of glyceollins on human prostate cancer cell LNCaP to further elucidate its potential effects on prostate cancer prevention. The glyceollins inhibited LNCaP cell growth similar to that of the soy isoflavone genistein. The growth inhibitory effects of the glyceollins appeared to be due to an inhibition on G1/S progression and correlated with an up-regulation of cyclin-dependent kinase inhibitor A1 and B1 mRNA and protein levels. By contrast, genistein only up-regulates cyclin-dependent kinase inhibitor A1. In addition, glyceollin treatments led to down-regulated mRNA levels for androgen responsive genes. In contrast to genistein, this effect of glyceollins on androgen responsive genes appeared to be mediated through modulation of an estrogen- but not androgen-mediated pathway. Hence, the glyceollins exerted multiple effects on LNCaP cells that may be considered cancer preventive and the mechanisms of action appeared to be different from other soy-derived phytochemicals.

### CROSS-REFERENCE TO RELATED APPLICATIONS

This Non-Provisional patent application claims the benefit of U.S. Provisional Patent Application No. 61/203,415, filed on 23 Dec. 2008, and which is hereby incorporated by reference in its entirety.

### BACKGROUND

1. Field

The present disclosure relates to increased biosynthesis and isolation of the isoflavonoid phytoalexin compounds, Glyceollins I, II, and III, in soy plants grown under stressed conditions, a composition containing said glyceollin(s), and methods of treating and preventing prostate cancer.

2. Description of Related Art

Prostate cancer is the most common non-cutaneous cancer among American men and is ranked third as a cause of cancer deaths. The number of new cases of prostate cancer and deaths due to prostate cancer is expected to increase dramatically over the next decade as baby boomer men age. If there is no change in prevention or treatment strategies, by 2015, there will be approximately 3 million men with prostate cancer, with about 45,000 deaths each year. Since there is currently no effective cure for this disease, there is much interest in developing preventive strategies to reduce prostate cancer's impact. Further, the available treatments for prostate cancer are serious and can have long-lasting effects.

Population and experimental studies have implicated dietary components in both the cause as well as prevention of prostate cancer. In particular, consumption of a diet that is rich in fruits, vegetables, and legumes is associated with a decreased risk for prostate and other forms of cancer. Hence, there is much interest in pursuing the development of food-derived products or compounds (e.g., phytochemicals) as chemopreventive agents due to their expected safety and the fact that they are perceived as supportive of medical therapies. The molecular targets of phytochemicals, as well as the mechanisms that contribute to their beneficial effects on cancer, remain elusive. Further elucidation of the molecular targets and mechanisms of phytochemicals would be important for exploiting their potential cancer preventive properties.

Of potential interest among diet-derived compounds are the isoflavones, including genistein and daidzein, which are found in soy products. The isoflavones are also known as phytoalexins. Phytoalexins constitute a chemically heterogeneous group of low molecular weight antimicrobial compounds that are synthesized de novo by plants and accumulate in response to stress. Soy contains several phytoalexins including the constitutive isoflavones daidzein and genistein that are considered as candidates for diet-derived prostate cancer preventive compounds. Initial interest in these compounds arose from studies that correlate consumption of soy products in Asian countries with a decreased incidence of hormone dependent cancers such as those of the mammary and prostate glands. Hence, a possible use for these compounds in mammary and prostate cancer chemoprevention has been suggested. Of the various soy-derived compounds, genistein has received the most interest due to its potent biological activity. Consumption of genistein has been shown to be protective against prostate cancer in animal models. The effect of genistein on various cellular pathways in cell culture models, including proliferation, apoptosis, cell cycle, and steroid hormone-mediated pathways has been reported. Genistein and daidzein can exert universal inhibitory effects on androgen responsive genes including prostate specific antigen (PSA) in the androgen responsive human prostate cancer cell LNCaP. The mechanisms by which genistein and daidzein exert their effects appeared to be through both androgen as well as estrogen receptor beta (ER-β) mediated events.

In addition to genistein and daidzein, the glyceollins represent another group of phytoalexins whose biosynthesis is increased in response to stress signals. The glyceollin isomers I-III (FIG. 1) have core structures similar to that of coumestrol (a natural derivative of coumarin) and are derived from the precursor daidzein. The glyceollins (I-III) can be derived naturally from exposure of soybean to the fungus Aspergillus sojae, a nontoxin-producing Aspergillus strain commonly used in the fermentation of soybeans to produce soy sauce and miso. Compared with genistein and daidzein, purified glyceollins show greater inhibition of estradiol's effects on proliferation and estrogen receptor (ER) signaling in breast cancer cells. Glyceollins also have enhanced antagonism toward ER-α relative to ER-β, and lack the estrogen agonist activity of genistein and daidzein seen in low-estrogen conditions. These findings suggest that soy protein enriched with glyceollins may have distinct estrogen-modulating properties compared with standard soy protein. The effects of the glyceollins toward prostate cancer remain unclear, but they may have similar activity towards human androgen responsive prostate cancer cells LNCaP.

Thus, there is a need to develop new treatments for prostate cancer from both synthetic and natural sources. Further, there is a need to reduce the risks of side effects as has been observed with treatments currently in use. Thus, in view of the universal inhibitory effects on androgen responsive genes including prostate specific antigen (PSA) in the androgen responsive human prostate cancer cell LNCaP in vitro and its lack of toxic activity, the efficacy of isolated glyceollins as a novel therapy in vivo was determined.

While certain novel features of this disclosure shown and described below are pointed out in the annexed claims, the disclosure is not intended to be limited to the details specified, since a person of ordinary skill in the relevant art will understand that various omissions, modifications, substitutions and changes in the forms and details of the disclosure illustrated and in its operation may be made without departing in any way from the spirit of the present disclosure. No feature of the disclosure is critical or essential unless it is expressly stated as being “critical” or “essential.”

### BRIEF SUMMARY

The present disclosure relates to glyceollins isolated from elicited soy which have been discovered to have universal inhibitory effects on androgen responsive genes including prostate specific antigen (PSA) in the androgen responsive human prostate cancer cell LNCaP. These glyceollins thus would be useful in the prevention and treatment of prostate cancer.

In accordance with this discovery, it is an object of the disclosure to provide isolated glyceollins (Glyceollin I, II, and III) from elicited soy.

It is a further object of the disclosure to provide a composition containing glyceollin for preventing or minimizing the development or growth of prostate cancer.

It is another object of the disclosure to provide a method for preventing or minimizing the development or growth of prostate cancer in a mammal, particularly a human.

Also part of this disclosure is a kit, comprising a glyceollin-containing composition for preventing or minimizing the development or growth of prostate cancer.

Further information on uses for glyceollins is disclosed in U.S. patent application Ser. No. 11/118,431, the disclosure of which is hereby incorporated by reference in its entirety.

In one embodiment, the present disclosure features a pharmaceutical composition comprising at least one isolated glyceollin for use in the treatment of prostate cancer. In said composition, the at least one isolated glyceollin may be present in an amount effective for the treatment of prostate cancer. Preferably, said at least one isolated glyceollin is isolated from elicited soy. Also preferably, the at least one isolated glyceollin isolated from elicited soy is Glyceollin I, II, III, or any combination thereof. In one aspect of this embodiment, the effective amount is selected on the basis of a treatment for prostate cancer. In another aspect of this embodiment, the effective amount is from 100 nM to 50 μM. In another aspect of this embodiment, the effective amount is from 1 mg/kg to 50 mg/kg. In another aspect of this embodiment, the composition is formed as a product for oral delivery, said product form being selected from a group consisting of a concentrate, dried powder, liquid, capsule, pellet, pill, and a food supplement including health bars. In another aspect of this embodiment, the composition is formed as a product for parenteral administration including intravenous, intradermal, intramuscular, and subcutaneous administration. In another aspect of this embodiment, the composition further comprises carriers, binders, diluents, and excipients.

In one embodiment, the present disclosure features a pharmaceutical composition comprising at least one isolated glyceollin for use in preventing, minimizing, or reversing the development or growth of prostate cancer in a male mammal. In said composition, the at least one isolated glyceollin may be present in an amount effective to prevent, minimize, or reverse the development or growth of prostate cancer in the mammal upon administration to said mammal. Preferably, said at least one isolated glyceollin is isolated from elicited soy. Also preferably, the at least one isolated glyceollin isolated from elicited soy is Glyceollin I, II, III, or any combination thereof. In one aspect of this embodiment, the effective amount is selected on the basis of a treatment for prostate cancer. In another aspect of this embodiment, the effective amount is from 100 nM to 50 μM. In another aspect of this embodiment, the effective amount is from 1 mg/kg/mammal to 50 mg/kg/mammal. In another aspect of this embodiment, the composition is formed as a product for oral delivery, said product form being selected from a group consisting of a concentrate, dried powder, liquid, capsule, pellet, pill, and a food supplement including health bars. In another aspect of this embodiment, the composition is formed as a product for parenteral administration including intravenous, intradermal, intramuscular, and subcutaneous administration. In another aspect of this embodiment, the composition further comprises carriers, binders, diluents, and excipients.

In one embodiment, the present disclosure features a method of inhibiting tumor growth comprising contacting a tumor with a composition comprising glyceollin and determining that growth of said tumor has been inhibited.

In one embodiment, the present disclosure features a method of preventing or treating cancer or tumor growth in a male individual comprising administering to the individual a composition comprising glyceollin and determining the development or growth of prostate cancer has been prevented, minimized, or reversed.

In one embodiment, the present disclosure features the use of at least one isolated glyceollin for the preparation of a medicament for treating a mammal suffering from or susceptible to prostate cancer. In one aspect of this embodiment, the at least one isolated glyceollin is present in the medicament in an amount effective for the treatment of prostate cancer. In another aspect of this embodiment, the at least one isolated glyceollin is isolated from elicited soy. In another aspect of this embodiment, the at least one isolated glyceollin isolated from elicited soy is Glyceollin I, II, III, or any combination thereof. In another aspect of this embodiment, the effective amount is selected on the basis of a treatment for prostate cancer. In another aspect of this embodiment, the effective amount is from 100 nM to 50 μM. In another aspect of this embodiment, the effective amount is from 1 mg/kg to 50 mg/kg.

Other objects and advantages of this disclosure will become readily apparent from the ensuing description.

### BRIEF DESCRIPTION OF THE DRAWINGS

FIG. 1 shows the structures of the soy isoflavone phytoalexins genistein, daidzein, glyceollin I, glyceollin II, and glyceollin III.

FIG. 2 demonstrates the effect of glyceollins and genistein on prostate cancer cell growth. LNCaP cells (0.25×10⁶ cells/well) were plated on 6-well plates. Cell treatments with varied concentrations (0-25 μM) of glyceollins or genistein were started 24 hours later for an additional 72 hours, and cell number determined as described below. * represent significantly different from control at p <0.05 (n=6).

FIG. 3 demonstrates the effect of glyceollins and genistein on prostate cancer cell growth. PC-3 cells (0.25×10⁶ cells/well) were plated on 6-well plates. Cell treatments with varied concentrations (0-25 μM) of glyceollins or genistein were started 24 hours later for an additional 72 hours, and cell number determined as described below. * represent significantly different from control at p<0.05 (n=6).

FIG. 4 demonstrates the effect of glyceollins on cell cycle in LNCaP cells. LNCaP cells (3×10⁶ cell) were plated in T-175 flask and treated with 0, 0.25, 2.5, 12.5, or 25 μM glyceollins or with and without genistein (25 μM) for 72 hr, cell cycle analysis performed as described below. Results are expressed as % of total cells (n=3).

FIG. 5 demonstrates the effect of genistein on cell cycle in LNCaP cells. LNCaP cells (3×10⁶ cell) were plated in T-175 flask and treated with 0, 0.25, 2.5, 12.5, or 25 μM glyceollins or with and without genistein (25 μM) for 72 hr, cell cycle analysis performed as described below. Results are expressed as % of total cells (n=3).

FIG. 6 is a representative histogram of effects of glyceollins (25 μM) on LNCaP cells. Histogram illustration of results for control cells treated with 25 μM glyceollins from FIGS. 4 and 5.

FIG. 7 is a representative histogram of effects of glyceollins (25 μM) on LNCaP cells. Histogram illustration of results for LNCaP cells treated with 25 μM glyceollins from FIGS. 4 and 5.

FIG. 8 demonstrates the effects of glyceollins and genistein on cell cycle in PC-3. PC-3 cells (3×10⁶ cells) were plated in T-175 flask and treated with or without 25 μM of glyceollins or genistein for 72 hr, cell cycle analysis performed as described below. Results are expressed as % of total cells (n=3).

FIG. 9 demonstrates the effect of glyceollins on CDKN1A mRNA levels. LNCaP cells cultured in 10% FBS were treated with 0, 2.5, 12.5, or 25 μM glyceollins for 48 h, total RNA isolated and mRNA levels of CDKN1A determined as described below. Results are expressed as mean +/−SD (n=3).

FIG. 10 demonstrates the effects of glyceollins on CDKN1B mRNA levels. LNCaP cells cultured in 10% FBS were treated with 0, 2.5, 12.5, or 25 μM glyceollins for 48 h, total RNA isolated and mRNA levels of CDKN1A determined as described below. Results are expressed as mean+/−SD (n=3).

FIG. 11 demonstrates the effects of glyceollins on CDKN1A and B protein levels. LNCaP cells were treated with and without 25 μM Glyceollins for 72 hours, cells were harvested and CDKN1A and B protein determined by western analysis as described below.

FIG. 12 demonstrates the effects of genistein on CDKN1A mRNA levels. LNCaP cells cultured in 10% FBS were treated with 0, 1, 5, 12.5 or 25 μM genistein for 48 h, total RNA isolated and mRNA levels of CDKN1A determined as described below. Results are expressed as mean+/−SD (n=3).

FIG. 13 demonstrates the effect of glyceollins on DHT- and 17β-estradiol-mediated growth in LNCaP cells. LNCaP cells (0.25×10⁶ cells/well) were plated in 6-well plate. Twenty-four hours later media was changed to Media B containing 10% CDS, followed 24 hours later with addition of varied concentrations (0-25 μM) of glyceollins in presence or absence of DHT (1 nM) or 17β-estradiol (10 nM) begin. Cells were treated for 72 hours and cell number determined as described in below. * represents significantly different from DHT or 17β-estradiol control at p<0.05 (n=6).

FIG. 14 demonstrates the effect of glyceollins on PSA mRNA levels in LNCaP cell cultured in 10% FBS. LNCaP cells cultured in 10% FBS were treated with 0, 2.5, 12.5, or 25 μM glyceollins for 48 h, total RNA isolated and mRNA levels of CDKN1A determined as described below. Results are expressed as mean+/−SD (n=3).

FIG. 15 demonstrates the effect of glyceollins and genistein on PSA protein levels in LNCaP cell cultured in 10% FBS. LNCaP cells cultured in 10% FBS were treated with or with 25 μM glyceollins or genistein for 72 hours, cell harvested and PSA protein determined using western analysis as described below.

FIG. 16 demonstrates the effect of glyceollins on DHT-induced increase in PSA mRNA levels. LNCaP cells were plated in 6-well plates; 24 h after plating the medium was switched to Media B, which contains 10% CDS, for an additional 24 h. Cells were then treated with or without DHT (1 nM) in the presence or absence of glyceollins (0, 2.5, 12.5, 25 μM) for 48 h, total RNA isolated, and mRNA levels of PSA were determined as described below. Results are expressed as mean+/−SD (n=3). Bars with different letters are significantly different from each other at p<0.05.

FIG. 17 demonstrates the effect of glyceollins on 17β-estradiol induced-increase in PSA mRNA levels. LNCaP cells were plated in 6-well plates; 24 h after plating the medium was switched to Media B, which contains 10% CDS, for an additional 24 h. Cells were then treated with or without 17β-estradiol (1 nM) in the presence or absence of glyceollins (0, 0.25, 2.5, 12.5, 25 μM) for 48 h, total RNA isolated, and mRNA levels of PSA were determined as described below. Results are expressed as mean+/−SD (n=3). Bars with different letters are significantly different from each other at p<0.05.

FIG. 18 demonstrates the effect of glyceollins on 17β-estradiol induced-increase in NKX3.1 mRNA levels. LNCaP cells were plated in 6-well plates; 24 h after plating the medium was switched to Media B, which contains 10% CDS, for an additional 24 h. Cells were then treated with or without 17β-estradiol (1 nM) in the presence or absence of glyceollins (0, 0.25, 2.5, 12.5, 25 μM) for 48 h, total RNA isolated, and mRNA levels of selected NKX3.1 were determined as described below. Results are expressed as mean+/−SD (n=3). Bars with different letters are significantly different from each other at p<0.05.

FIG. 19 demonstrates the effect of glyceollins on 17β-estradiol induced-increase in IGF-IR mRNA levels. LNCaP cells were plated in 6-well plates; 24 h after plating the medium was switched to Media B, which contains 10% CDS, for an additional 24 h. Cells were then treated with or without 17β-estradiol (1 nM) in the presence or absence of glyceollins (0, 0.25, 2.5, 12.5, 25 μM) for 48 h, total RNA isolated, and mRNA levels of IGF-1R were determined as described in Materials and Methods. Results are expressed as mean+/−SD (n=3). Bars with different letters are significantly different from each other at p<0.05.

### DETAILED DESCRIPTION

Detailed descriptions of one or more preferred embodiments are provided herein. It is to be understood, however, that the present disclosure may be embodied in various forms. Therefore, specific details disclosed herein are not to be interpreted as limiting, but rather as a basis for the claims and as a representative basis for teaching one skilled in the art to employ the present disclosure in any appropriate manner.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

This disclosure describes the increased biosynthesis of the isoflavonoid phytoalexin compounds, Glyceollins I, II and III, in soy plants grown under stressed conditions (elicited soy) and their marked effects on estrogen-modulated pathway function. To fully understand the role of glyceollins' anti-estrogenic, anti-androgenic and anti-cancer properties, we used the well-established model of LNCaP and PC-3 human prostate cancer cells in an in vitro model to examine the effects of glyceollins on cell growth. In this model, using the LNCaP and PC-3 human prostate cancer cells, the in vitro anti-androgenic activity of the glyceollins has been established.

As used herein, the term “ER” refers to “estrogen receptor”. The term “prostate cancer” refers to any cancer having its origin in prostate cells, and includes metastatic and local forms of prostate cancer. The term “minimize” or “reduce”, or a derivative thereof, includes a complete or partial inhibition of a specified biological effect (which is apparent from the context in which the term minimize is used). The term “glyceollin” may mean both a single glyceollin and plural glyceollins when the glyceollin is defined as at least one of a selected group of glyceollins.

The glyceollin compounds used in the compositions and methods of the present disclosure are naturally occurring substances which may be found in plants such as soybeans that are stressed or that have been treated with elicitors. The glyceollin compounds may be isolated from the plant sources in which they naturally occur after treatment with an elicitor, or may be synthetically prepared by processes known in the art.

It is preferred to extract the glyceollins useful in the compositions and methods of the present disclosure from the plant materials in which they naturally occur. A preferred method of isolating the glyceollin compounds is to extract the plant materials with an alcohol, preferably methanol or ethanol, or an aqueous methanolic solution, to remove the glyceollins from the plant material. It is preferred to comminute the plant material before extracting the glyceollin compounds to maximize recovery of glyceollin compounds from the plant material. The glyceollin compounds are isolated from the extract by conventional separation procedures, such as high performance liquid chromatography, HPLC.

In a preferred embodiment, the glyceollin compounds are isolated from a soy material. Soy materials from which the glyceollin compounds can be isolated include elicitor-treated: soy seeds, soybeans, dehulled soybeans, soy cotyldeons, soy leaf tissue, soy roots, and soy hypocotyls. In one embodiment, the glyceollins are extracted from soy seeds, with a low molecular weight organic extractant, preferably an alcohol, ethyl acetate, acetone, or ether, and most preferably aqueous ethyl alcohol or methyl alcohol.

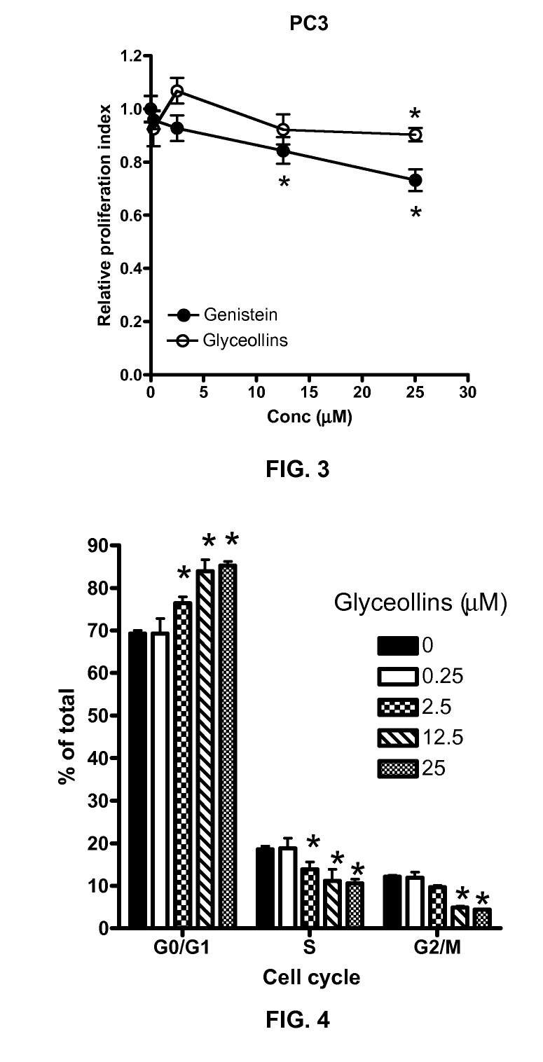

Here we demonstrate that specific glyceollins, isolated from elicited soy, displayed anti-androgenic activity, significantly inhibiting LNCaP cell growth in a concentration-dependent manner. The inhibitory effects of the glyceollins can be observed at 2.5 μM. The growth inhibitory effects of the glyceollins on LNCaP cells were similar to that observed for genistein (FIG. 2). However, in the androgen non-responsive prostate cancer cell PC-3 (FIG. 3), the effects of glyceollins was attenuated. Similar attenuated effects were also observed for genistein in PC-3 cells (FIG. 3).

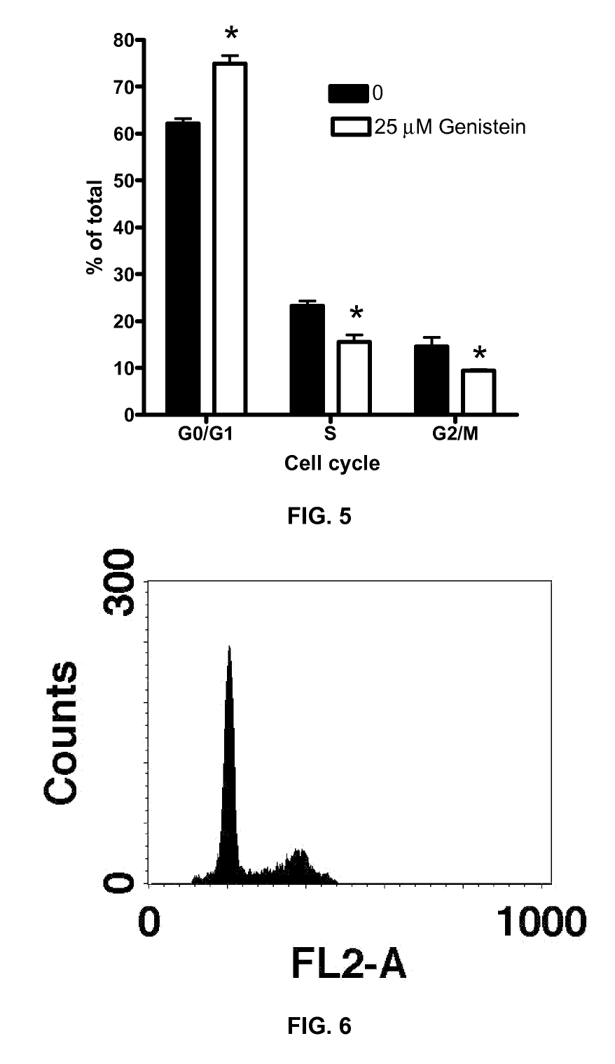

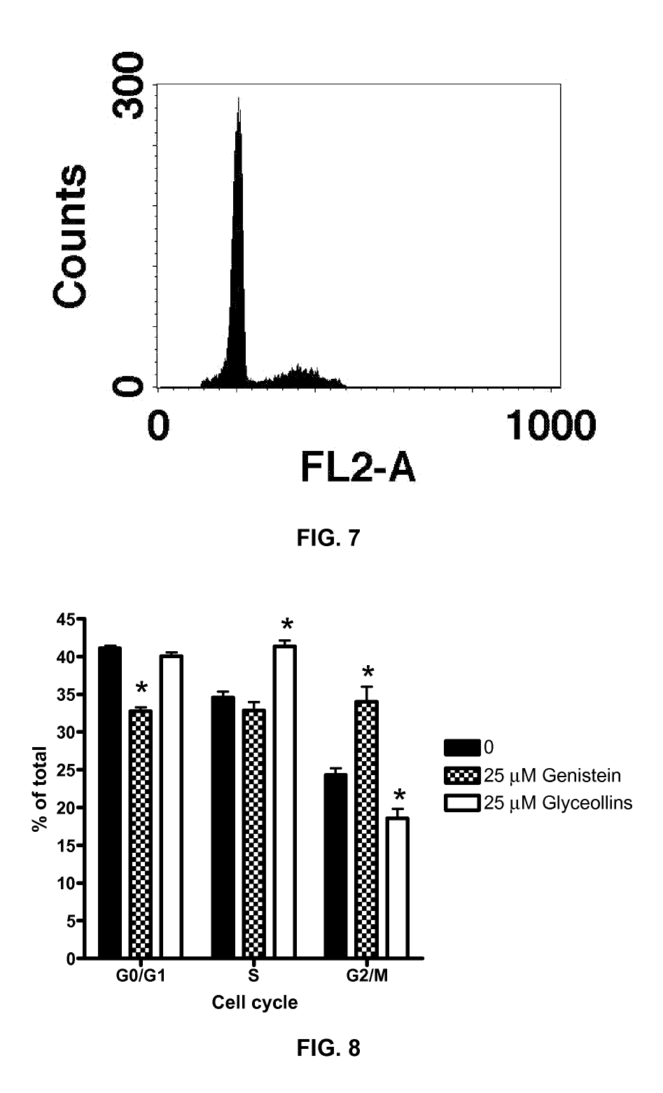

To further elucidate the mechanism(s) by which the glyceollins treatment resulted in growth inhibition, the effects of the glyceollins on cell cycle progression was also examined. As shown in FIG. 5 and FIG. 6, treatment of LNCaP cells with the glyceollins for 72 hours led to concentration-dependent effects on G1/S arrest. Similarly, treatment of LNCaP cell with genistein (25 μM) for 72 hours also leads to G1/G0 arrest (FIG. 4). By contrast, genistein (25 μM) and not glyceollins (25 μM) treatments for 72 hours lead to G2/M blockage in PC-3 cells (FIG. 8). Glyceollins treatment appeared to lead to S phase blockages in PC3 Cells (FIG. 8). The cell cycle analysis did not reveal any significant effects of the glyceollins on apoptotic events as indicated by lack of sub-2N PI staining of DNA (FIG. 6 and FIG. 7). Additionally, induction of the caspase 3/7 activation in glyceollins treated LNCaP cells was not observed.

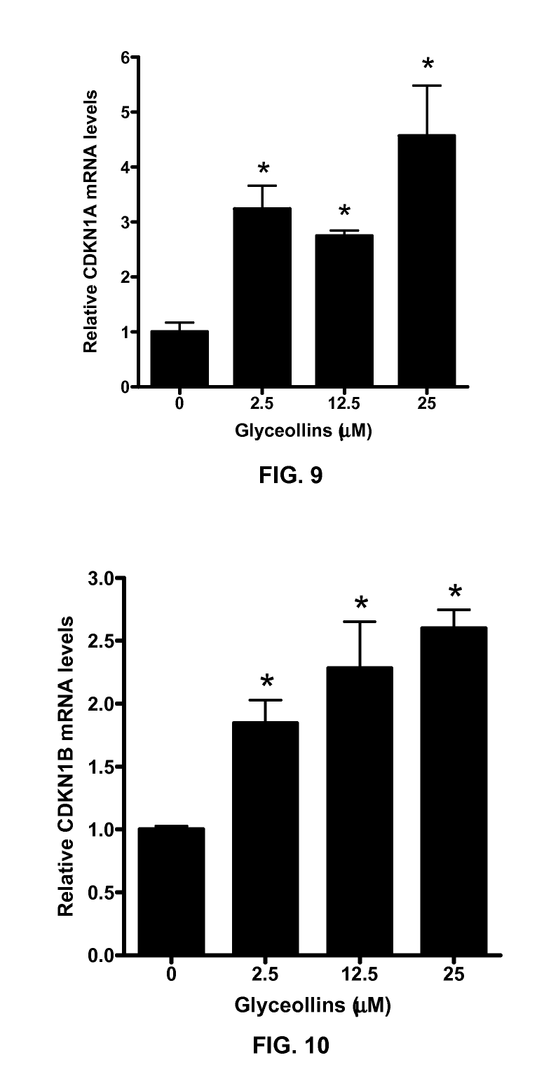

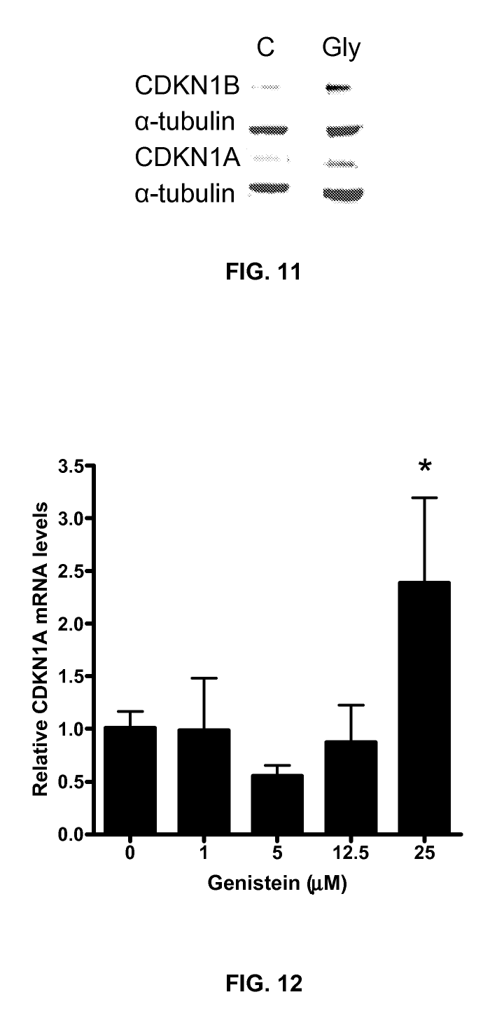

The cyclin-dependent kinase inhibitors CDKN1A and B mRNA expression are modulated during cell cycle progression and are involved in G1/S arrest. To gain additional perspective at the molecular levels of the glyceollins growth inhibitory effects, the effects of the glyceollins on CDKN1A and B mRNA levels in LNCaP cells was also determined. As shown in FIG. 9 and FIG. 10, after 48 hours treatment glyceollin appeared to induce both CDKN1A and B mRNA levels. There were significant changes at 2.5 μM for both CDKN1A and B mRNA levels. Up regulation of these cyclin inhibitors were confirmed at the protein level (FIG. 11). By contrast an induction of CDKN1A mRNA by genistein was only observed at 25 μM (FIG. 12), there were no changes in CDKN1B mRNA levels in LNCaP cells treated with genistein at all concentrations (0-25 μM) tested. Consistent with lack of effect of the glyceollins on apoptosis, no alteration was detected in either Bax or Bcl-2, two well documented regulators of apoptosis pathways, mRNA expression.

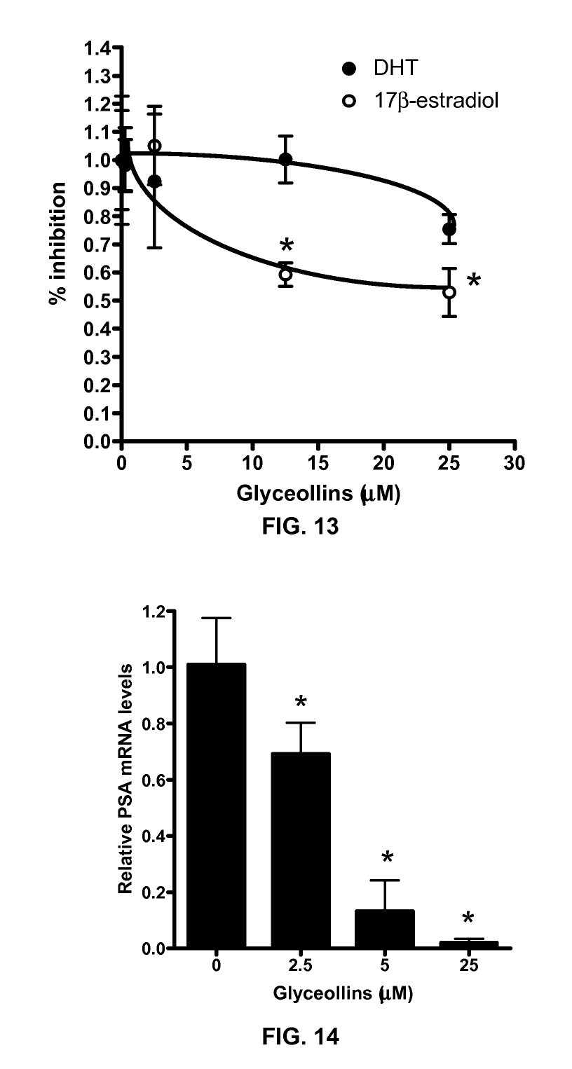

Prostate cancer LNCaP cell growth can be subject to modulation by androgen as well as estrogen. To further identify proximal events modulated by the glyceollins that result in cell cycle arrest and growth inhibition, the effects of the glyceollins on DHT (1 nM) and 17β-estradiol (10 nM) induced LNCaP cell growth were examined. The concentration of steroid hormones was chosen based on their physiological achievable levels as well as in-vitro efficacy. As shown in FIG. 13, after 72 hr treatment of LNCaP cells with the glyceollins led to an inhibition of 17β-estradiol-induced growth, but not DHT-induced growth of LNCaP cells.

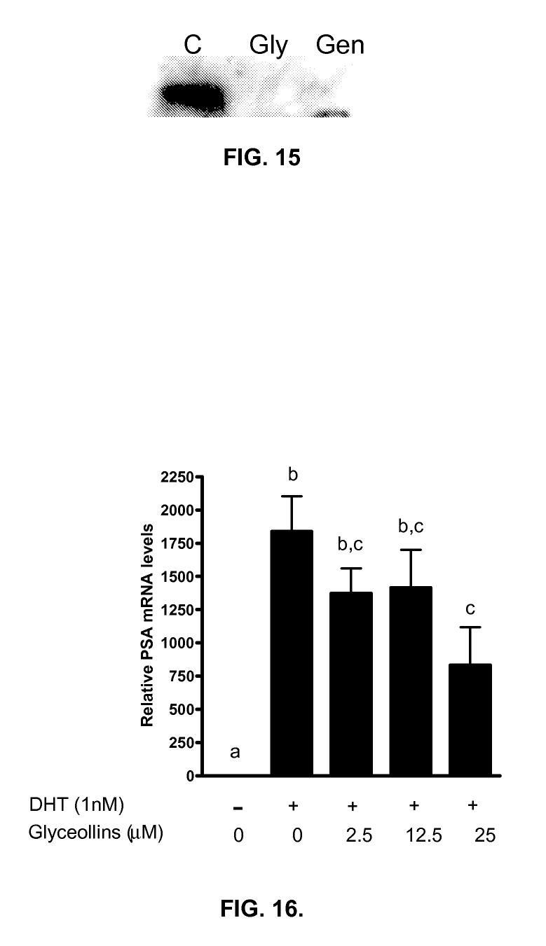

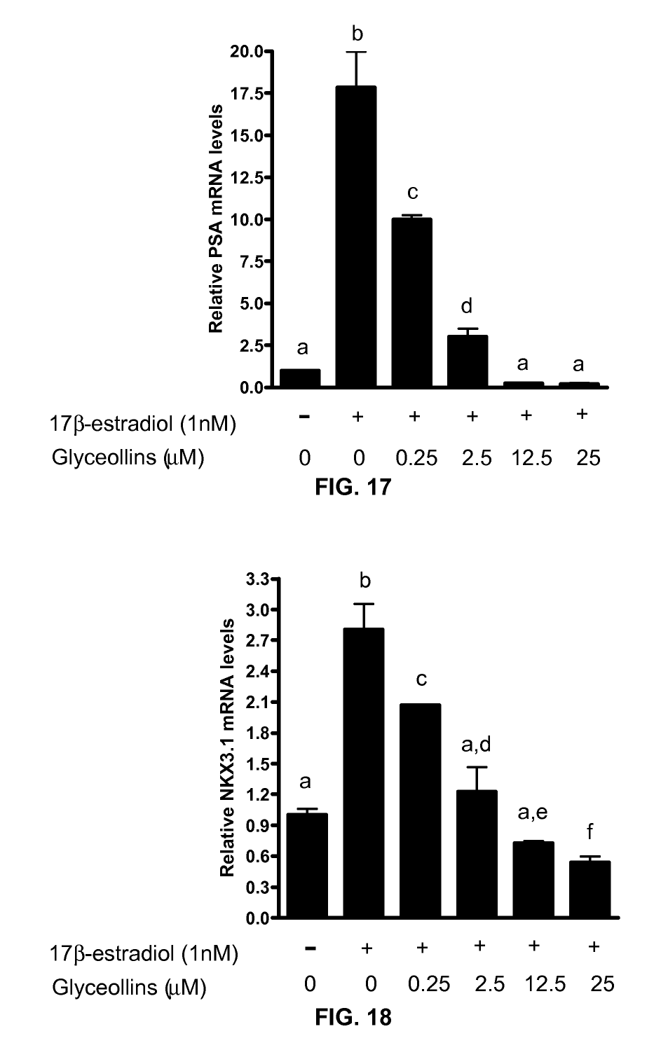

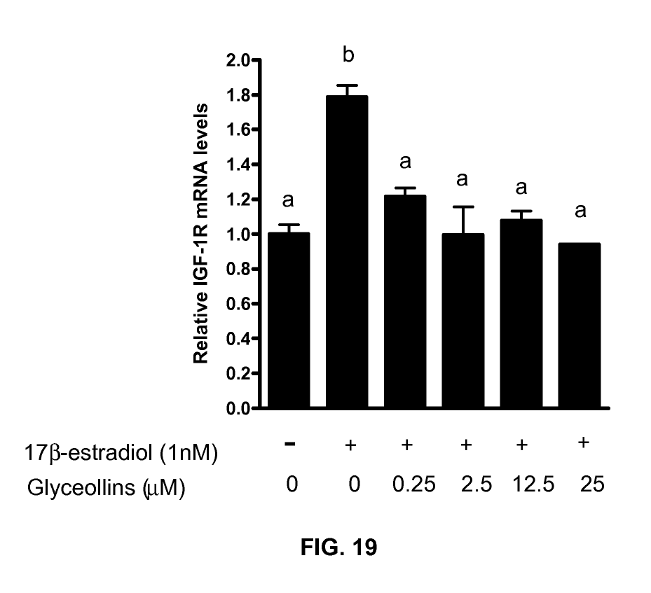

It has been previously shown that the androgen responsive genes PSA not only respond to androgen, but also to 17β-estradiol through ER-β-mediated events. Hence, the effects of glyceollins on this gene were examined as a surrogate end point to further elucidate the effects of the glyceollins on the androgen- and estrogen-responsive pathways. As shown in FIG. 14 and FIG. 15, glyceollins treatment of LNCaP cell culture in 10% FBS for 48 hours led to an attenuation of PSA mRNA and protein levels supporting the hypothesis of possible hormonal regulation. As shown in FIG. 16, the glyceollins appeared not to be effective in inhibiting the DHT-induced increase in PSA mRNA levels. It required at least 25 μM of glyceollins to produce a significant inhibition of DHT-induced PSA mRNA levels. In contrast, the glyceollins effectively inhibited the 17β-estradiol-induced increase in PSA mRNA levels (FIG. 17). The effect can be seen starting at a concentration of 0.25 μM. Furthermore the inhibition of 17β-estradiol-induced increase in two other androgen/estrogen responsive genes, NKX3.1 (FIG. 18) and IGF-IR (FIG. 19) mRNA levels, was also observed. The concentration effects of glyceollins on these two genes were similar to the effect on PSA mRNA and occur at 0.25 μM.

### Materials and Methods

Chemicals. Dihydrotestosterone (DHT), dimethylsulfoxide (DMSO), and genistein, 17β-estradiol were from Sigma Chemical Co. (St. Louis, Mo.). Cell culture media and reagents were purchased from Invitrogen (Carlsbad, Calif.).

Soybean treatment and harvesting. Aspergillus sojae (SRRC 1125) cultures were grown at 25° C. in the dark on potato dextrose agar. After 5 days, inoculum was prepared by harvesting conidia (3.4×10⁷/ml) in 15 ml sterile, distilled H₂O. Seeds from commercial soybean variety Asgrow 5902 were surface-sterilized for 3 min in 70% ethanol followed by a quick deionized-H₂O rinse and two 2 min rinses in deionized-H₂O. Seeds were presoaked in sterile deionized-H₂O for 4-5 hr, and then chopped for 2 min in a Cuisinart food processor. Aspergillus sojae spore suspension (300 ml) was applied to the cut surface of seeds on each tray. All trays were stored at 25° C. in the dark for three days, rinsed with water to remove spores, and oven dried at 40° C. for 24 hrs. Seeds were ground using a Waring blender before extraction.

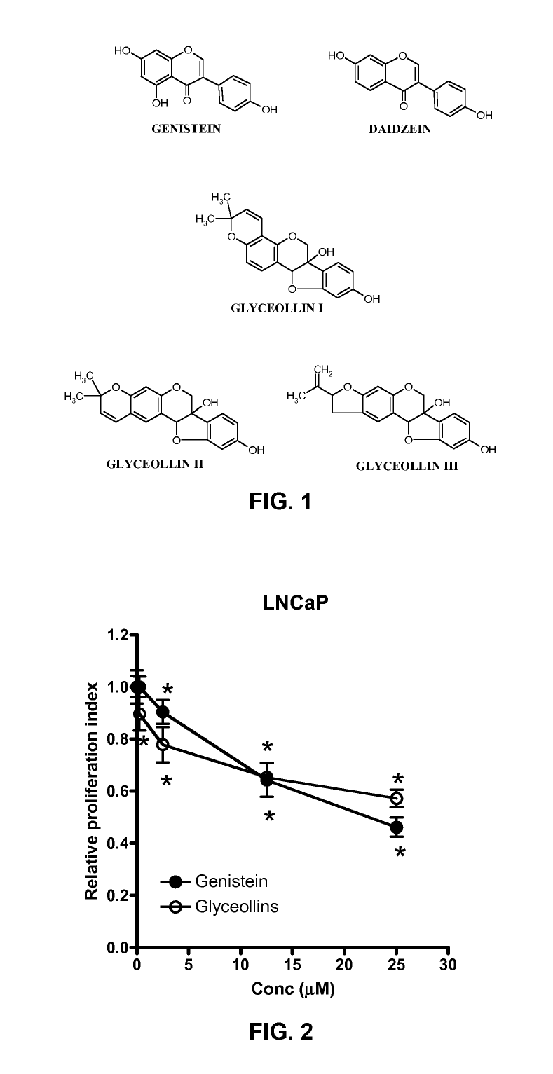

Isolation of glyceollins (I-III). The glyceollins I, II, and III were extracted from the 300 g ground seeds with 1 L methanol. The glyceollins were isolated using preparative scale HPLC using two Waters 25 mm 10 mm particle size mBondapak C18 radial compression column segments combined using an extension tube. HPLC was performed on a Waters 600E System Controller combined with a Waters UV-VIS 996 detector. Elution was carried out at a flow rate of 8.0 ml/min with the following solvent system: A=acetonitrile, B=water; 5% A for 10 min, then 5% A to 90% A in 60 min followed by holding at 90% A for 20 min. The injection volume was 20 mL. The fraction containing the glyceollins was concentrated under vacuum and freeze-dried. The glyceollins were confirmed by UV-VIS spectrophotometry, mass spectrometry, and NMR. The solvents acetonitrile (HPLC grade) and methanol were purchased from Aldrich Chemical Company. Water was obtained using a Millipore system and used during sample preparation procedures and HPLC analyses. A mixture of glyceollins I (68%), II (21%), and III (11%) were isolated (see FIG. 1) and used in treatments. An average MW of 338 was use to calculate the concentration of glyceollins used in all cell culture experiments.

Cells and cell culture. LNCaP and PC-3 human prostate cancer cells were obtained from the American Type Culture Collection (Manassas, Va.) and maintained in Media A [RPMI 1640 medium with phenol red (Invitrogen, Carlsbad, Calif.), 2 mM L-glutamine (Sigma), 100 U/mL penicillin and 100 μg/mL streptomycin (BioSource International, Camarillo, Calif.) with 10% fetal bovine serum (FBS) (Invitrogen, Carlsbad, Calif.)]. Cells were incubated in the presence of 5% CO₂ in air at 37° C.

Cell growth assay. LNCaP or PC-3 cells (5×10⁴ cells/well) were plated in 24-well plates (Costar); treatments were begun 24 h later. Cells were treated with 0, 1, 5, 10 or 25 μM glyceollins or genistein (DMSO as vehicle) for 0-72 h, and the medium containing test compound was replaced every 24 h. Cell growth was analyzed using the sulforhodamine B (SRB) assay. For experiments using the DHT or 17β-estradiol, cells were switched to Media B [RPMI 1640 medium without phenol red, 2 mM L-glutamine (Sigma), 100 U/mL penicillin and 100 μg/mL streptomycin with 10% charcoal dextran-treated FBS (CDS, Hyclone, Logan, Utah)] 24 h after plating to minimize the effect of serum hormones. The cells were then incubated in Media B for an additional 24 hours before the treatments were begun.

Cell cycle analysis using flow cytometry. LNCaP or PC3 cells (3×10⁶ cells) were seeded into T-175 flask in Medium A. Twenty-four hours later the medium was changed to that containing vehicle or test compounds. Concentration dependent effects of glyceollins (0-25 μM was studied in LNCaP cell. In PC-3 cell, comparisons were made between cells treated with or without 25 μM glyceollins. For genistein, comparisons were made between cell treated with or without 25 μM genistein in both LNCaP and PC-3 cells. Cells were treated for 72 hours and harvested, transferred into centrifuge tubes (50 mL polypropylene) pellet (1000×g), wash 1× in PBS (no Ca or Mg) and pelleted again. Cell pellets were then re-suspended in 1.5 mL PBS. To re-suspended cells, 15 mL of 70% ethanol was added and the capped tubes vortexed gently. The ethanol fixed cells were pelleted and washed one time in PBS. Washed cells were fixed in ethanol and stained for DNA content using propidium iodide (PI). The cellular DNA was then analyzed by flow cytometry. DNA content of the cells was determined through flow cytometry using a FACScalibur cytometer (Becton Dickinson, San Jose, Calif.). Flow cytometric data files were collected and analyzed using the CELLQuest program (Becton Dickinson). A total of 10,000 cell events were collected for DNA analyses. Cell cycle distribution percentages of stained nuclei were calculated using Modfit LT software (Version 3.0, Verity Software House, Inc., Topsham, Me.). Calibration standards (LinearFlow Green and DNA QC Particle Kit) for verification of instrument performance were purchased from Molecular Probes (Eugene, Oreg.) and Becton Dickinson, respectively.

Determination of the effects of glyceollins on gene expression in LNCaP cells using RT-PCR. To examine the effects of glyceollins on cyclin inhibitor CDKN1A and B mRNA expression, LNCaP cells were plated in 6-well plates (0.25×10⁶ cells/well) in Media A. After twenty-four hours the medium was removed and replaced with fresh medium containing vehicle, 1, 5, or 25 μM glyceollins or genistein. For experiments examining the effects of glyceollins on steroid hormone, LNCaP cells were plated in 6-well plates (0.25×10⁶ cells/well) in Media A and switched to Media B containing 10% CDS 24 h after plating to minimize the effect of serum hormones. Twenty-four hours later, the medium was replaced with fresh medium containing 1 nM DHT or 17β-estradiol with or without 0-25 μM glyceollin. For all experiments fresh medium containing the test compounds was changed daily and cells were harvested for total RNA isolation using the Trizol method (Invitrogen) after 48 h. Taqman real-time PCR was used to quantify expression of the mRNA. Taqman real-time PCR Primer and probes for glyceraldehydes-3-phosphate dehydrogenase (G3PDH), PSA, cyclin-dependent kinase inhibitor (CDKN)1A and CDKN1B, NKX3.1(NK3 homeobox 1), Bcl-2, Bax and insulin like growth factor-1 receptor (IGF-1R) were purchased form Applied Biosystems (Foster City, Calif.).

Apoptosis assay. Activation of caspase was used as an additional method to flow cytometry to detect apoptosis. LNCaP cells (1×10⁶ cells/well) were plated in 6-well plates and 24 hrs later the glyceollins (25 μM final concentration) were added. After 72 hrs of treatment with or without the test compounds, cells were washed with PBS once and lysed in cell lysis buffer (Biosource, Camarillo, Calif.). Protein was determined using the BCA method (Pierce, Rockford, Ill.) according to manufacturer's protocol. Fifty μg of lysate was used for determination of caspase activity using the Caspase-Glo 3/7 Assay (Promega, Madison, Wis.) following manufacturer's protocol. Western Blots. LNCaP cells were plated in 100 mm×20 mm cell culture dish in Media A and switched to Media B containing 10% CDS 24 hours after plating to minimize the effect of serum hormones. Twenty-four hours later, the medium was replaced with fresh medium containing vehicle (DMSO) or 25 μM glyceollins. Fresh medium containing the test compound was changed daily and cells were harvested for Western Blot analysis after 72 hours of treatments. The lysed extracts were collected, than centrifuged at 10,000×g for 10 minutes. The supernatants were used to determine the protein concentration. Following this, the supernatant, sample buffer, and reducing agent were added and the samples were heated at 105° C. and loaded onto a 4-12% gradient SDS-PAGE gel (Invitrogen, Carlsbad, Calif.). Gels were then transferred to nitrocellulose membranes and the membranes were probed with mouse anti-p21^{Waf1/Cip1} and rabbit anti-p27^{Kip1} at a 1:1000 dilution as primary antibodies (Cell Signaling, Danvers, Mass.) followed by incubation with IR-tagged secondary antibodies (LiCor Biosciences, Lincoln, Neb.). The blots were analyzed using the Odyssey Infrared Imaging System (LiCor Biosciences, Lincoln, Neb.).

Statistics. All treatments were repeated at least 3 times and representative experiments were presented. Experimental data were analyzed using the Prism 4 statistical software package (GraphPad software.). Unpaired t tests were used for two group comparisons. For multiple group comparisons, ANOVA followed by post hoc analysis using Bonferroni's test were employed. Treatments effects with a p value of <0.05 were considered significant.

The present disclosure is further illustrated by the following examples. These examples are provided to aid in the understanding of the disclosure and are not construed as a limitation thereof.

### EXAMPLE 1

### Effect of Glyceollins and Genistein on Prostate Cancer Cell Growth

To examine the effects of glyceollins on prostate cancer prevention, we first tested the effects of glyceollin on LNCaP cell growth. As shown in FIG. 2, after 72 hours treatment, glyceollins inhibited LNCaP cell growth in a concentration-dependent manner. The inhibitory effects of the glyceollins can be observed at 2.5 μM. The growth inhibitory effects of the glyceollins on LNCaP cells were similar to that observed for genistein (FIG. 2). However, in the androgen non-responsive prostate cancer cell PC-3 (FIG. 3), the effects of glyceollins was attenuated. Similar attenuated effects were also observed for genistein in PC-3 cells (FIG. 3). To further elucidate the mechanism(s) by which the glyceollins treatment resulted in growth inhibition, we also examined the effects of the glyceollins on cell cycle progression. As shown in FIG. 5, FIG. 6 and FIG. 7, treatment of LNCaP cells with the glyceollins for 72 hours led to concentration-dependent effects on G1/S arrest. Similarly, treatment of LNCaP cell with genistein (25 μM) for 72 hours also leads to G1/G0 arrest (FIG. 4). By contrast, genistein (25 μM) and not glyceollins (25 μM) treatments for 72 hours lead to G2/M blockage in PC-3 cells (FIG. 8). Glyceollins treatment appeared to lead to S phase blockages in PC3 Cells (FIG. 8). The cell cycle analysis did not reveal any significant effects of the glyceollins on apoptotic events as indicated by lack of sub-2N PI staining of DNA (FIG. 6 and FIG. 7). Additionally, we also did not observe induction of the caspase 3/7 activation in glyceollins treated LNCaP cells.

### EXAMPLE 2

### Effect of Glyceollins on the Modulation of Cyclin-Dependent Kinase Inhibitors mRNA Levels in LNCaP Cells

The cyclin-dependent kinase inhibitors CDKN1A and B mRNA expression are modulated during cell cycle progression and are involved in G1/S arrest. To gain additional perspective at the molecular levels of the glyceollins growth inhibitory effects, we also determined the effects of the glyceollins on CDKN1A and B mRNA levels in LNCaP cells. As shown in FIG. 9 and FIG. 10, after 48 hours treatment glyceollin appeared to induce both CDKN1A and B mRNA levels. There were significant changes at 2.5 μM for both CDKN1A and B mRNA levels. Up regulation of these cyclin inhibitors were confirmed at the protein level (FIG. 11). By contrast we only observed an induction of CDKN1A mRNA by genistein at 25 μM (FIG. 12), there were no changes in CDKN1B mRNA levels in LNCaP cells treated with genistein at all concentrations (0-25 μM) tested. Consistent with lack of effect of the glyceollins on apoptosis, we also did not detect alteration in either Bax or Bcl-2, two well documented regulators of apoptosis pathways, mRNA expression.

### EXAMPLE 3

### Effect of Glyceollins on Androgen- and Estrogen-Induced Growth

Prostate cancer LNCaP cell growth can be subject to modulation by androgen as well as estrogen. To further identify proximal events modulated by the glyceollins that result in cell cycle arrest and growth inhibition, we examined the effects of the glyceollins on DHT (1 nM) and 17β-estradiol (10 nM) induced LNCaP cell growth. The concentration of steroid hormones was chosen based on their physiological achievable levels as well as in-vitro efficacy. As shown in FIG. 13, after 72 hr treatment of LNCaP cells with the glyceollins led to an inhibition of 17β-estradiol-induced growth, but not DHT-induced growth of LNCaP cells.

### EXAMPLE 4

### Effects of Glyceollins on Androgen-and Estrogen Induced Genes mRNA Levels in LNCaP Cells

We have previously shown that the androgen responsive genes PSA not only respond to androgen, but also to 17β-estradiol through ER-β-mediated events. Hence, we examined the effects of glyceollins on this gene as a surrogate end point to further elucidate the effects of the glyceollins on the androgen- and estrogen-responsive pathways. As shown in FIG. 14 and FIG. 15, glyceollins treatment of LNCaP cell culture in 10% FBS for 48 hr led to an attenuation of PSA mRNA and protein levels supporting the hypothesis of possible hormonal regulation. This similar to that of genistein on PSA mRNA we reported earlier and protein (FIG. 15). We also ask whether this effect of glyceollins is through an androgen- or estrogen-dependent pathway. As shown in FIG. 16, the glyceollins appeared not to be effective in inhibiting the DHT-induced increase in PSA mRNA levels. It required at least 25 μM of glyceollins to produce a significant inhibition of DHT-induced PSA mRNA levels. In contrast, the glyceollins effectively inhibited the 17β-estradiol-induced increase in PSA mRNA levels (FIG. 17). The effect can be seen starting at a concentration of 0.25 μM. Furthermore we also observed inhibition of 17β-estradiol-induced increase in two other androgen/estrogen responsive genes, NKX3.1 (FIG. 18) and IGF-1R (FIG. 19) mRNA levels. The concentration effects of glyceollins on these two genes were similar to the effect on PSA mRNA and occur at 0.25 μM.

What is claimed is:

1. A pharmaceutical composition comprising at least one isolated glyceollin for use in the treatment of prostate cancer.

2. The composition of claim 1, wherein the at least one isolated glyceollin is present in an amount effective for the treatment of prostate cancer.

3. The composition of claim 2, wherein said at least one isolated glyceollin is isolated from elicited soy.

4. The composition of claim 3, wherein said at least one isolated glyceollin isolated from elicited soy is Glyceollin I, II, III, or any combination thereof.

5. The composition of claim 2, wherein the effective amount is selected on the basis of a treatment for prostate cancer.

6. The composition of claim 2, wherein said effective amount is from 100 nM to 50 μM.

7. The composition of claim 2, wherein said effective amount is from 1 mg/kg to 50 mg/kg.

8. A pharmaceutical composition comprising at least one isolated glyceollin for use in preventing, minimizing, or reversing the development or growth of prostate cancer in a male mammal.

9. The composition of claim 8, wherein said at least one isolated glyceollin is present in an amount effective to prevent, minimize, or reverse the development or growth of prostate cancer in the mammal upon administration to said mammal.

10. The composition of claim 9, wherein said at least one isolated glyceollin is isolated from elicited soy.

11. The composition of claim 10, wherein said at least one isolated glyceollin isolated from elicited soy is Glyceollin I, II, III, or any combination thereof.

12. The composition of claim 9, wherein the effective amount is selected on the basis of a treatment for prostate cancer.

13. The composition of claim 9, wherein said effective amount is from 100 nM to 50 μM.

14. The composition of claim 9, wherein said effective amount is from 1 mg/kg/mammal to 50 mg/kg/mammal.

15. The composition of claim 1, in which said composition comprises a product for oral delivery, said product form being selected from a group consisting of a concentrate, dried powder, liquid, capsule, pellet, pill, and a food supplement including health bars.

16. The composition of claim 1, in which said composition comprises a product for parenteral administration including intravenous, intradermal, intramuscular, and subcutaneous administration.

17. The composition of claim 1, further comprising carriers, binders, diluents, and excipients.

18. A method of inhibiting tumor growth comprising contacting a tumor with a composition comprising glyceollin and determining that growth of said tumor has been inhibited.

19. A method of preventing or treating cancer or tumor growth in a male individual comprising administering to the individual a composition comprising glyceollin and determining that the development or growth of prostate cancer has been prevented, minimized, or reversed.

20. A method of treating a mammal suffering from or susceptible to prostate cancer, comprising providing to said mammal at least one isolated glyceollin.

21. The method of claim 20, wherein the at least one isolated glyceollin is present in a medicament in an amount effective for the treatment of prostate cancer.

22. The method of claim 21, wherein said at least one isolated glyceollin is isolated from elicited soy.

23. The method of claim 22, wherein said at least one isolated glyceollin isolated from elicited soy is Glyceollin I, II, III, or any combination thereof.

24. The method of claim 21, wherein the effective amount is selected on the basis of a treatment for prostate cancer.

25. The method of claim 21, wherein said effective amount is from 100 nM to 50 μM.

26. The method of claim 21, wherein said effective amount is from 1 mg/kg to 50 mg/kg.
